(19) United States
(12) Patent Application Publication
(10) Pub. No.: US 2024/0410872 A1
(43) Pub. Date: Dec. 12, 2024

(54) METHOD AND SYSTEM FOR SOIL-MOISTURE MONITORING

(71) Applicant: ALMA MATER STUDIORUM - UNIVERSITA' DI BOLOGNA, Bologna (IT)

(72) Inventors: Matteo FRANCIA, Bologna (IT); Enrico GALLINUCCI, Bologna (IT); Joseph GIOVANELLI, Bologna (IT); Matteo GOLFARELLI, Bologna (IT); Elena BALDI, Bologna (IT); Maurizio QUARTIERI, Bologna (IT); Moreno TOSELLI, Bologna (IT)

(21) Appl. No.: 18/689,384

(22) PCT Filed: Sep. 8, 2022
(86) PCT No.: PCT/IB2022/058461

§ 371 Date: Mar. 5, 2024

(30) Foreign Application Priority Data
Sep. 8, 2021 (IT) 102021000023162

(51) Int. Cl. G01N 33/24; G06N 20/00

(52) U.S. Cl. CPC G01N 33/246; G06N 20/00

(57) ABSTRACT

A method for estimating a quantity profile of a volume of soil involves placing in the soil volume a bi-dimensional or three-dimensional grid of quantity sensors, each quantity sensor of the grid being identified by a position represented by three-dimensional coordinates with respect to a center of the soil volume, acquiring, in a same instant of time, a quantity value from each quantity sensor, so as to obtain a bi-dimensional or three-dimensional grid of real quantity values, and applying a profiling function to the quantity value grid to obtain a fine grained quantity profile defined by a bi-dimensional or three dimensional extended grid of quantity values. Each value of the extended grid is associated with a position represented by three-dimensional coordinates with respect to the center of the soil volume. The number of elements of the extended grid of quantity values is much greater than the number of quantity sensors.

The present disclosure relates to methods and systems for monitoring quantities that are present in a volume of soil through a grid of sensors placed in the soil.

Nowadays many quantities of the soil can be precisely measured using different types of sensors, such as for example water potential, or moisture, (using Gypsum blocks or Watermarks sensors), soil temperature, electrical conductivity and, more recently, Nitrate concentrations (see, for example, T. Turkeltaub, D. Kurtzman, O. Dahan, “Real-time monitoring of nitrate transport in the deep vadose zone under a crop field-implications for groundwater protection”, Hydrology and Earth System Sciences 20 (8) (2016) 3099-3108).

Although the method and system according to the present disclosure can be applied to different quantities and in different applicative contexts (e.g. agriculture, geology, construction), here and after we will focus on soil moisture in smart agricolture to keep the discussion focused and fluent.

Controlling the soil moisture is a crucial factor in optimizing watering and crop performance. For example, Kiwi (Actinidia deliciosa) has high water demand (average daily evapotranspiration ranging from 62 m³/day in New Zealand to 80 and 103 m³/day in the central zone of South America). Indeed, Kiwi is commonly an irrigated crop in most of the areas where it is cultivated.

Different types of watering systems may be adopted depending on the farming features and needs: dripper (single and double wings), sprinkler, hosereel and flood irrigation. In orchards, where a stable watering system can be built, drip irrigation is widely used as it enables precise watering which reduces water waste.

The risks of over-watering range from groundwater depletion to suffocation of the plant. As to kiwi, farmers tend to over-watering since it leads to larger fruits, but this reduces their dry mass and jeopardizes their maintenance after harvest. Ideally, the moisture level should be known and optimal on the whole soil volume taken by roots. Such volume is subject to strong horizontal and vertical variability caused by many related factors: (a) uneven root suction, (b) limited watering covering, (c) difference in the soil layers in terms of composition, exposure to atmospheric agents and compression, (d) punctual anomalies of the ground (e.g., hysteresis).

The monitoring of the Soil Moisture (hereinafter “SM”) has been proven to represent beneficial in many various applications. In agriculture, based on the adopted watering technique, different analyses have been conducted. Publication Tu et al., “Water budget components estimation for a mature citrus orchard of southern china based on hydrus-1d model”, Agricultural Water Management 243, investigates the water demand of a mature citrus orchard of southern China to optimize the available water budget during rainy and dry seasons. Liang et al., “Adaptive prediction of water droplet infiltration effectiveness of sprinkler irrigation using regularized sparse autoencoder-adaptive network-based fuzzy inference system (rsae-anfis)”, Water (Switzerland) 13 (6), predicts the droplet infiltration effectiveness of sprinkler irrigation in order to improve accuracy and efficiency.

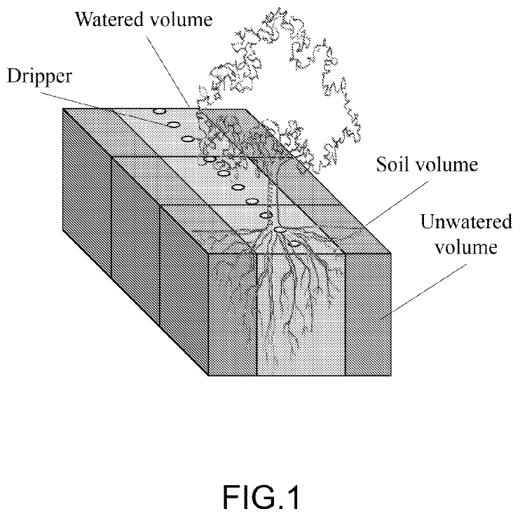

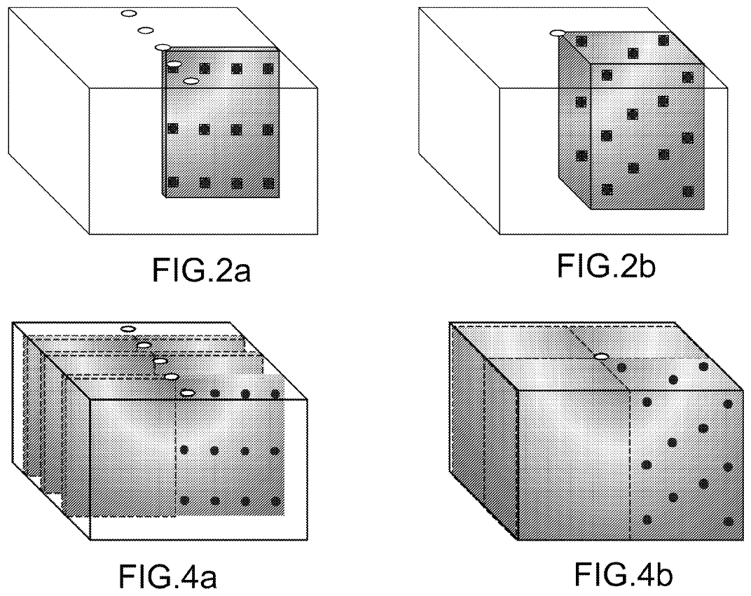

FIG. 1 shows an example of an orchard watered through a single wing dripper line. The cubes show the soil volume taken by the roots. On the one hand, the limited distance between drippers ensures homogeneous moisture along the row of trees (i.e., watered volume); on the other hand, the effect of watering is reduced by moving on the inter-row and a portion of the soil volume remains completely unwatered (i.e., unwatered volume). In this case, a 2D grid such as the one reported in FIG. 2a is sufficient to create a comprehensive profile. Conversely, when relevant moisture variations take place along the row of trees too, a 3D grid, as in FIG. 2b, is more suited. Variations on the row of trees dimension may be determined, for example, by too sparse drippers or by non-homogeneous suction of the roots.

Previous approaches building a soil profile exploit sensors to carry out inverse calibration of a simulation model (e.g., J. Simunek, M. Van Genuchten, M. Sejna, “Development and applications of the hydrus and stanmod software packages and related codes”, Vadose Zone Journal 7 (2) (2008) 587-600) and thus (a) requires a long-times series of data from the field; (b) binds the profile exploitation to an expensive and resource-consuming simulator; (c) requires frequent updates.

For these reasons, those approaches are typically used for carrying out spot researches to study soil moisture dynamics. There is the need to provide a cost-effective, operative solution to monitor soils moisture whose reference application is watering optimization.

The present disclosure is aimed at creating a fine-grained multidimensional profile of a soil quantity which is validated by sensors.

Indeed, for moisture monitoring, previous approaches build a soil profile either by (i) training machine learning algorithms atop synthetic data or by (ii) exploiting sensors to carry out inverse calibration of a numerical model. As to (i), using synthetic data only leads to theoretical models which are not representative of actual soil moisture profiles and which, without considering sensors at all, are not validated on actual data. As to (ii), inverse calibration of numerical models requires: long time series of data from the field, an expensive and resource-consuming simulator, and frequent (seasonal) re-calibrations. For these reasons, those approaches are typically used for carrying out spot researches to study soil moisture dynamics.

Conversely, the object of the present disclosure is to provide a cost-effective operative solution to monitor soil quantities, for example soils moisture.

Therefore, one object of the present invention is to create a quantity profile that is representative of the whole soil volume.

A particular object of the present invention is to preserve an optimal moisture level without wasting water.

Such objects are achieved with a method for estimating a quantity profile of a volume (V) of soil according to claim 1 and with a system according to claim 10. The dependent claims describe preferred or advantageous embodiments of the method.

According to one aspect of the invention, a method for estimating a quantity profile of a volume (V) of soil is proposed. In a general embodiment, the method comprises the steps of:
placing in the soil volume a bi-dimensional or three-dimensional grid of quantity sensors, in which each sensor of the grid is identified by a position represented by three-dimensional coordinates with respect to the center of the soil volume;
acquiring, in the same instant of time, a quantity value from each quantity sensor of the sensor grid, to obtain a bi-dimensional or three-dimensional grid of real quantity values;
applying a profiling function to the quantity value grid in order to obtain a fine grained quantity profile defined by a bi-dimensional or three dimensional extended grid of quantity values, each value of the extended grid being associated with a position represented by three-dimensional coordinates with respect to the center of the soil volume, wherein the number of elements of the extended grid of quantity values is much greater than the number of quantity sensors.

In one embodiment, the profiling function is a linear interpolation function between pairs of actual quantity values.

In particular, the linear interpolation function may be applied between the pairs of real quantity values along each axis of the quantity value grid, independently of the other axes.

In one embodiment, the linear interpolation is a bi-linear or a three-linear interpolation.

According to this bi-linear or three-linear interpolation, each interpolated value of the extended grid is calculated considering the rectangle or parallelepiped whose vertices are the real quantity values closest to the point of the interpolated value and calculating a first linear interpolation between the pairs of real quantity values along a first dimension of the rectangle or parallelepiped, in order to obtain a pair of interpolated quantity values, a second linear interpolation along the second dimension of the rectangle or parallelepiped between the pairs of interpolated quantity values, and a possible third linear interpolation along the third dimension of the parallelepiped.

In an alternative embodiment, the profiling function is a non-linear function that takes into account the characteristics of the soil.

In principle, any numerical model or machine-learning algorithm that estimates the measured quantity exploiting the specific soil features can be adopted. As to machine learning methods, for example, regression trees and Support Vector Machines.

For example, in one embodiment related to moisture monitoring, the profiling function may be implemented by means of a machine learning algorithm based on an artificial neural network of the “feed-forward” type with a single hidden layer, wherein the input layer has a neuron for each sensor. The output layer has as many neurons as the elements of the approximate grid of moisture values.

In one embodiment, the machine learning algorithm may include the steps of:
generation of a training data set, including the calibration of a numerical model that simulates the hydrogeological flows of the soil in many and different weather and irrigation conditions, the training data set representing fine grid moisture profiles;
learning of a profiling function that maps real moisture values obtained from the sensors into a fine grid moisture profile.

According to another aspect of the invention, a system for estimating a quantity profile of a volume (V) of soil is proposed. The system comprises:
a bi-dimensional or three-dimensional grid of quantity sensors that can be positioned in the soil volume, in which each sensor of the grid is identified by a position represented by three-dimensional coordinates with respect to the center of the soil volume;
a processing unit operatively connected to the sensor grid.

The processing unit is programmed to acquire, in the same instant of time, a quantity value from each quantity sensor of the sensor grid, so as to obtain a bi-dimensional or three-dimensional grid of real values of the quantity, and to apply a profiling function to the quantity value grid in order to obtain a fine grained quantity profile defined by a bi-dimensional or three dimensional extended grid of quantity values, each value of the extended grid being associated with a position represented by three-dimensional coordinates with respect to the center of the soil volume, wherein the number of elements of the extended grid of quantity values is much greater than the number of quantity sensors.

Therefore, in the present disclosure, the inventors propose:
a 2D/3D quantity profile based on a grid of quantity sensors. The grain of the profile is in the order of magnitude of square/cubic centimeters;
two alternative solutions to estimate a fine-grained profile by adopting linear and non-linear approximations. In particular, non-linear approximation relies on machine learning algorithms to learn the soil model providing soil texture;
an analysis of the trade-off between the accuracy and the cost of the plant varying the number and the position of sensors;
original profile visualizations that enable visual exploitation of the profile, for example 2D and 3D profile visualizations, profile average and profile variance over a time-lapse.

Further features and advantages of the method and system according to the invention will become apparent from the following description of preferred embodiments thereof, given by way of non-limiting, indicative example, with reference to the accompanying figures, wherein:

FIG. 1 is a schematic perspective view of the relevant elements in an orchard;

FIGS. 2a and 2b show regular 2D and 3D sensor grids, respectively;

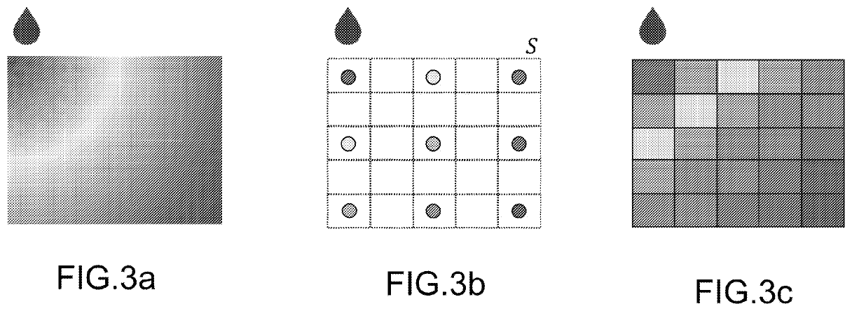

FIGS. 3a, 3b, and 3c are snapshots of moisture in a soil slice, where FIG. 3a represents actual soil moisture, FIG. 3b is the raw sensor grid, and FIG. 3c represents the soil profile;

FIGS. 4a and 4b represent symmetries in watered soil, in case of 2D and 3D grid, respectively;

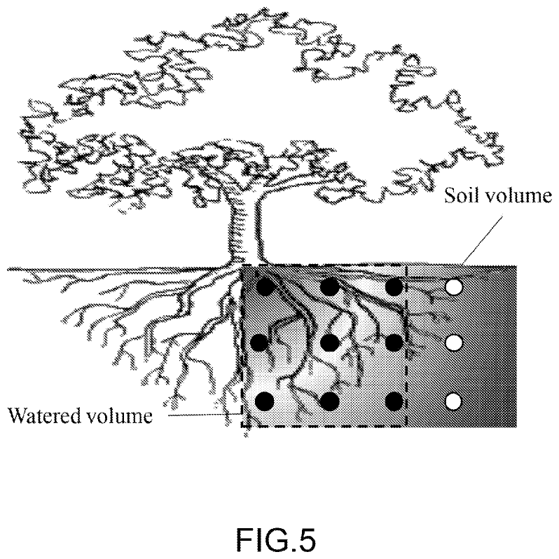

FIG. 5 is a sensor layout to monitor watered and unwatered volumes;

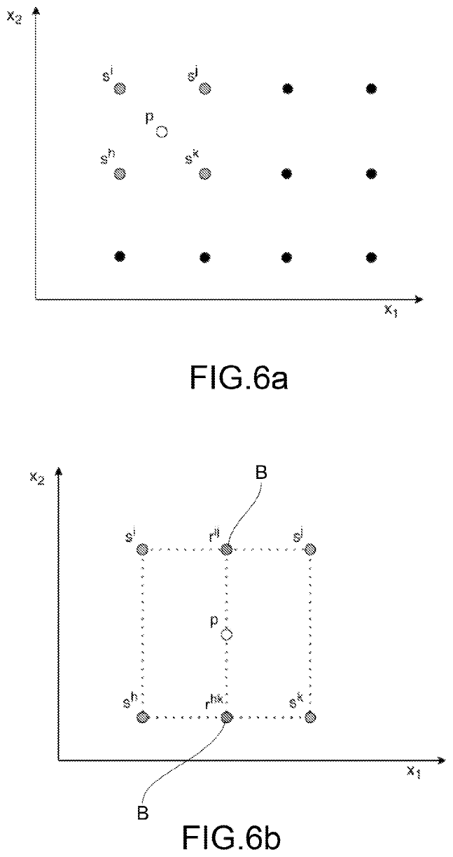

FIGS. 6a and 6b are diagrams representing two steps of a 2D example for the Soil-Feature Unaware interpolation algorithm;

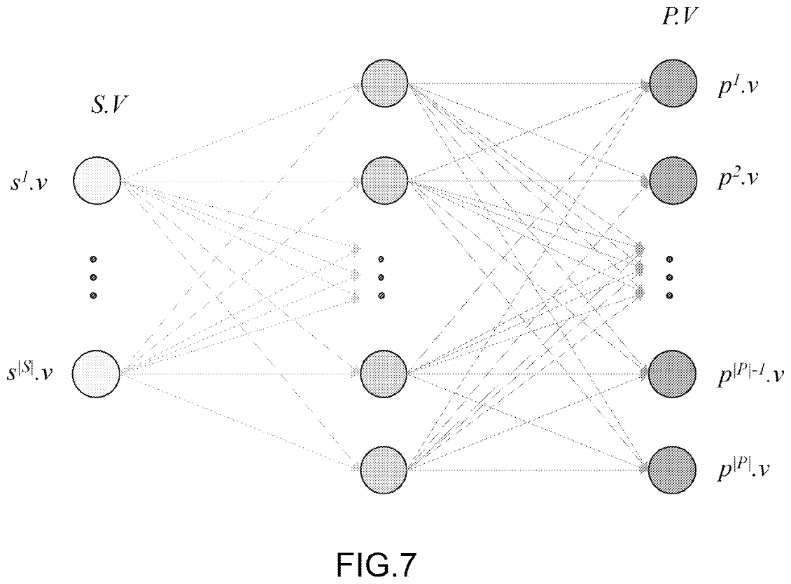

FIG. 7 is a layout example of an ANN profile function;

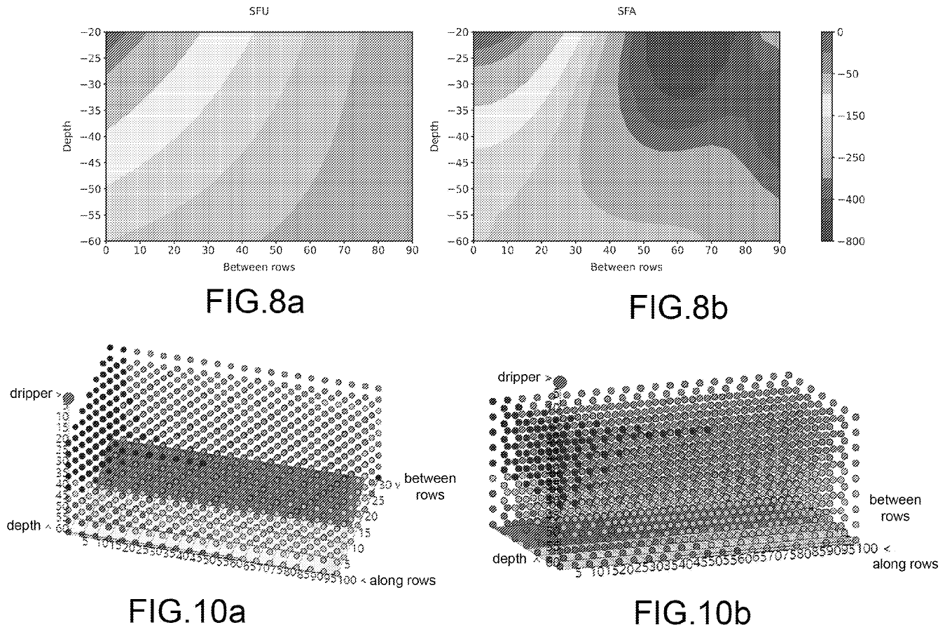

FIGS. 8a and 8b are examples of moisture profiles built by SFU (8a), and built by SFA (8b);

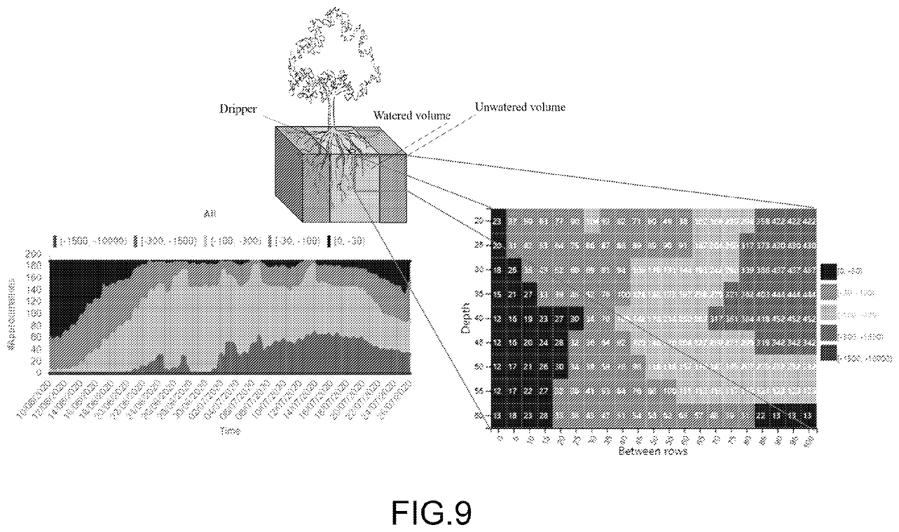

FIG. 9 is a 2D-visualization example of historical and spatial moisture trends;

FIGS. 10a and 10b are two perspective views of a 3D spatial moisture chart;

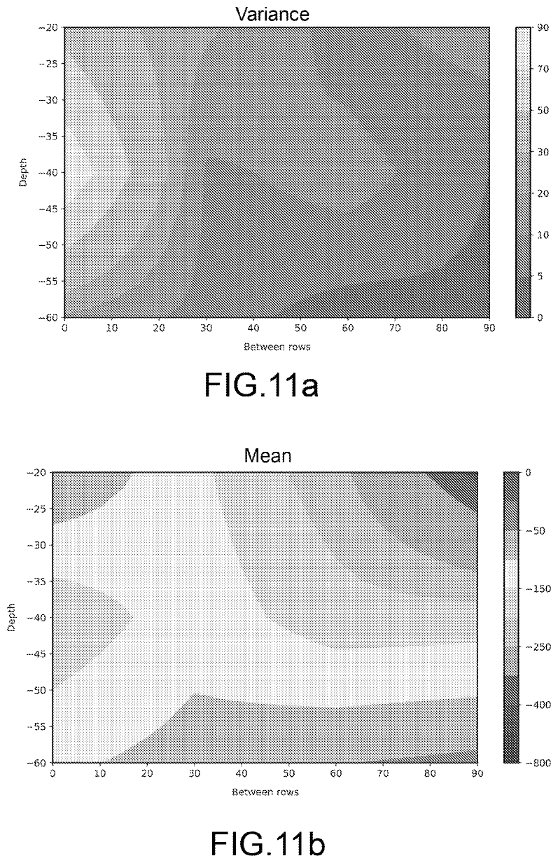

FIGS. 11a and 11b shows the variance and mean profile charts, respectively, of the moisture (in cbar) along a given period;

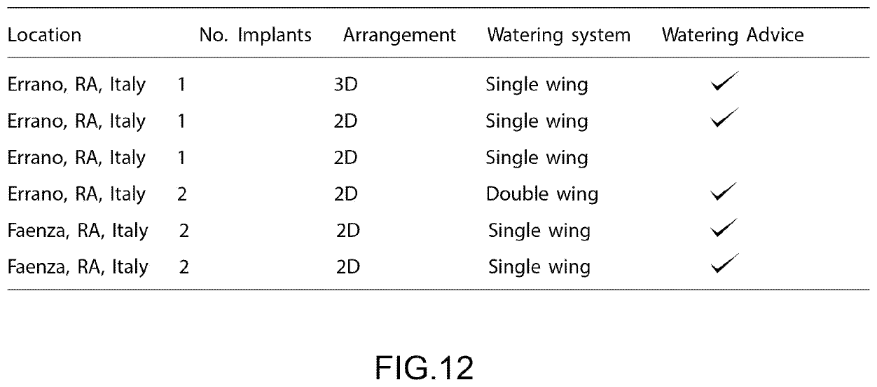

FIG. 12 is a table listing some implants arrangement used for the tests;

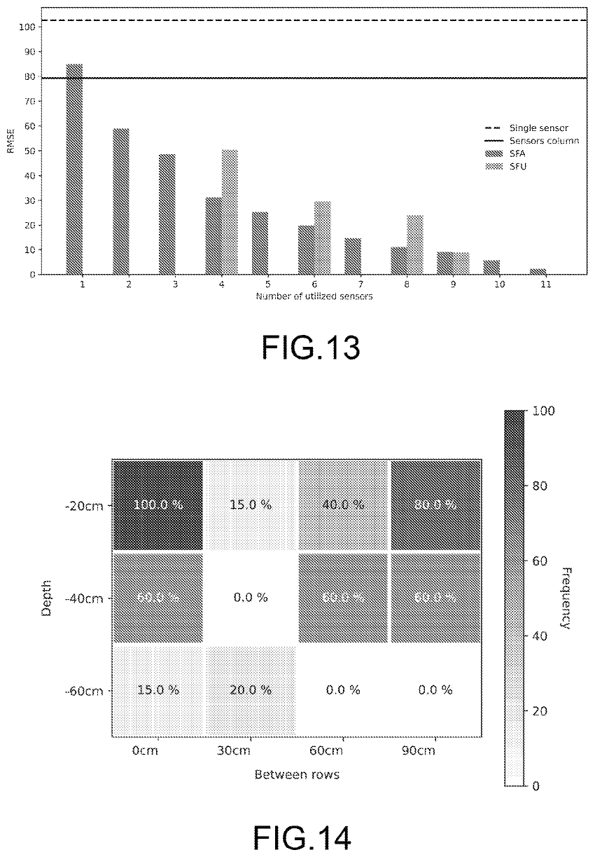

FIG. 13 is a diagram representing the performance of two profiling functions in comparison to the agriculture standard approaches;

FIG. 14 is a diagram representing the frequency of times each sensor appeared in the best arrangements;

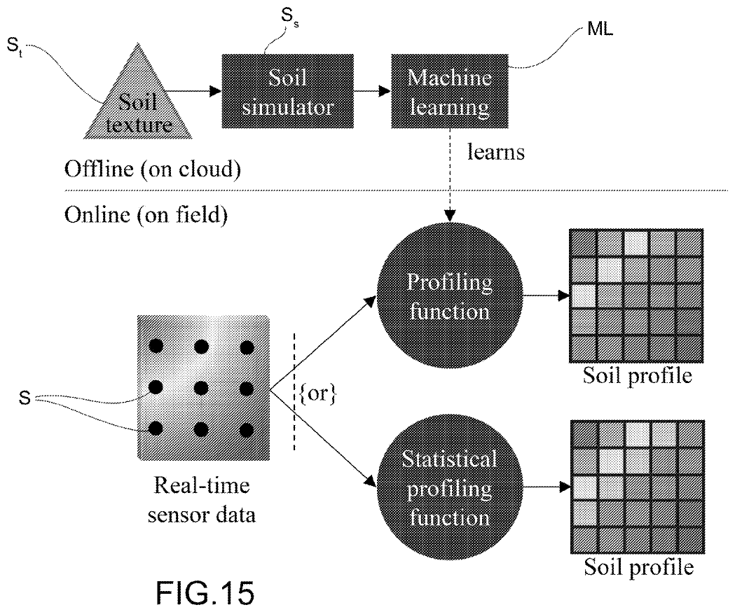

FIG. 15 is a diagram of an example of a system architecture implementing the methods according to the invention; and

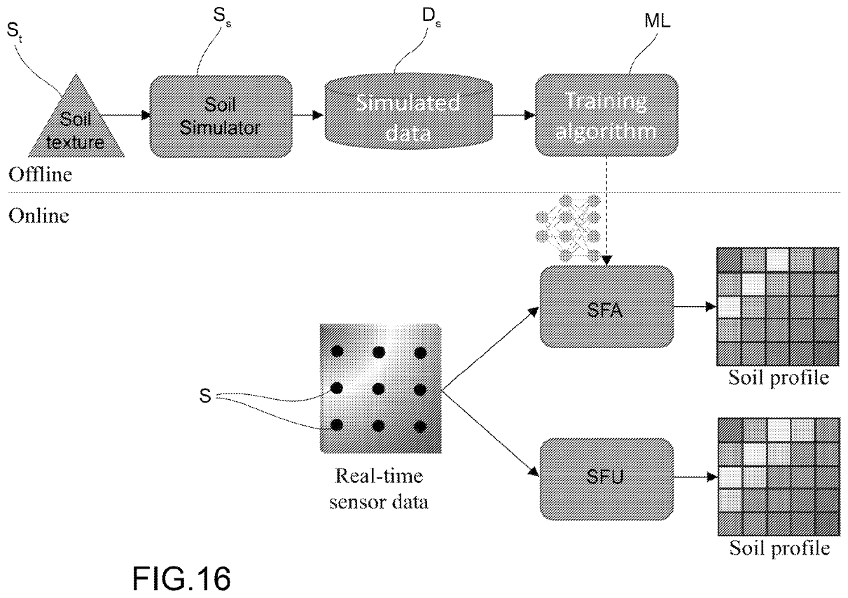

FIG. 16 shows an embodiment variant of the system architecture of FIG. 15.

In the following description, some examples of methods and systems for estimating the moisture of a volume of soil will be illustrated, referring to the definitions below.

Definition 1 (Soil volume). Given a plant, its soil volume is a parallelepiped of soil that contains most of the plant roots.

Definition 2 (Sensor grid). A sensor grid S={s¹, . . . , s^{|s|}} is a n-dimensional layout of |S| sensors installed in a soil volume. Each sensor sⁱ is defined by a three-dimensional displacement {sⁱ.x₁, sⁱ.x₂, sⁱ.x₃} with respect to the center of the soil volume. A grid measure S.V is a set of sensor values collected at the same time: S.V={s¹.v, . . . , s^{|s|}.v}.

Depending on n, the grid can resemble a rectangle (n=2) and a parallelepiped (n=3). The value monitored depends on the type of sensor in the context of smart watering, soil moisture is the water content of the soil expressed, for instance, in terms of volume or pressure.

The proposed soil moisture monitoring method can operate with any sensor that provides a numerical measurement of soil moisture regardless of the technology used. For example: sensors that measure water potential (sensors based on gypsum block belong to this category), sensors that measure the volumetric percentage of water in the ground (FDR, “Reflectometry in the frequency domain”, sensors and TDR, “Time domain reflectometry” sensors belong to this category). Soil moisture measurement can therefore be based on in-situ volumetric or potential sensors such as gypsum blocks. In one example of installation, the orchard is watered through a single wing dripper line; moisture is monitored through a 2D sensor grid of 12 sensors.

Moisture varies continuously within the soil volume while the raw sensors provide point-wise measurements.

Moisture profile, based on raw sensor measurements, estimates moisture of the whole soil volume at a fine-grained resolution.

Definition 3 (Moisture profile). Given a n-dimensional sensor grid S the moisture profile is a n-dimensional grid P={p¹, . . . , p^{|P|}} that approximates, in each pⁱ, the soil moisture measured by S. The set of approximated moisture values is P.V={p¹.v, . . . , p^{|P|}.v}. P is fine-grained with reference to S since |P|>|S|. The approximation pⁱ.v is assumed to be constant in the region surrounding pⁱ, whose granularity depends on |P|.

For example, if |S|=12 and |P|=1000 the soil granularity is 5 squared/cubic cm depending on the profile dimensionality. FIG. 3c shows a sensor grid with |S|=9 and a moisture profile with |P|=25.

Moisture profile covers the sensor grid region that is typically smaller, in size and in dimensionality, than the soil volume. To provide a comprehensive estimation of the whole soil volume, we rely on the following soil assumptions:
Symmetry: the portions of the soil volume that are not covered by the sensor grid have the same behavior as the monitored one, that is, they are symmetric with reference to the moisture dynamics.
Stationarity: in the unwatered volume (see FIG. 1) moisture does not depend on the distance from the drippers. Moisture variations are limited in this volume and mainly change according to the depth.

FIGS. 4a and 4b show the symmetries respectively in the case of 2D and 3D grids.

The symmetry assumption is verified for 2D grids when a reduced distance of the drippers ensures homogeneous moisture along the cultivation row. If the distance between the drippers is high and the moisture along the row is not constant, to satisfy the symmetry assumption a 3D grid must be adopted.

The previous considerations allow us to extend the moisture profile to the whole soil volume provided that the sensor grid layout: (a) covers an area that fulfills the symmetric assumption; (b) has a sufficient number of sensors to fulfill the stationary assumption.

FIG. 5 shows a layout that enables the whole soil volume to be characterized: black sensors stand in the watered area, while white ones cover moisture variations in the unwatered volume. The stationary makes it simpler to comprehensively monitor the unwatered region: a 1d column of sensors at different depths, placed in the unwatered volume is sufficient to monitor the whole region.

The transformation of raw sensor measurements (FIG. 3b) into a moisture profile (FIG. 3c) is achieved through a profiling function.

Definition 4 (Profiling function). Given a n-dimensional sensor grid S and a moisture profile P, a profiling function f(S)=P computes the moisture profile values.

According to this approach, I corresponds to sensors located in the same positions as the ones from the real sensor grid, while O represents the sensors from the soil profile, for instance, a uniform sensor grid where adjacent sensors are at 5 cm distance. Given I and 0, two different implementations are proposed.

The role of a profiling function is to approximate the moisture values in those positions of the moisture profile where a sensor is not present. A profiling function is based on sensor grid measurements, and it can optionally exploit further information about the behavior of the soil. Several possible profiling functions can be adopted. In this disclosure two alternative approaches are proposed, that differ in the information exploited:
Soil-feature unaware—SFU: this is a statistical profiling function that exploits the sensor measurements only. The most obvious choice is to carry out a linear interpolation between pairs of sensor values.
Soil-feature aware—SFA: this is a profiling function with the aid of an offline learning phase, which exploits the knowledge about the hydrological fluxes in the soil to keep into account non-linearities and to get a better estimate.

On the one hand, a SFU function is general purpose and does not require to be fitted to a specific field. On the other hand, a SFA function results in a more accurate moisture profile. This allows also the SFU approaches to capture non-linear behaviors. Although local regression approaches are bounded to linear behavior between sensor pairs, the composition of several linear strokes approximates a non-linear trend. Conversely, SFA approaches model the non-linearities between sensor pairs too. The gap between the two approximations grows as the distance between the sensors increases.

As to the SFU function, we rely on the well-known n-linear interpolation, where n is the profile and grid dimensionality. For the sake of conciseness, in the following, we describe the 2-linear case. Given a 2D sensor regular grid S, BLin(sⁱ, s^{j}, s^{h}, s^{k}) carries out a linear interpolation in each dimension independently from the others. The approach consists of two phases (see FIGS. 6a and 6b).

For each point p€P of the moisture profile to be computed: (i) we find the four sensors {sⁱ, sⁱ, s^{h}, s^{k}}€S that determine the minimum bounding rectangle including p (FIG. 6a), then (ii) we compute p:v=BLin(sⁱ, sⁱ, s^{h}, s^{k}) (see FIG. 6b). The interpolation along the x₁ axis is considered first (dots B): r^{ij}.v (with r^{ij}.x₁=p.x₁ and r^{ij}.x₂=sⁱ.x₂=s^{j}.x₂) is obtained as the linear interpolation of sⁱ.v and s^{j}.v; the same holds for r^{hk}.v (with r^{hk}.x₁=p.x₁ and r^{hk}.x₂=s^{h}.x₂=s^{k}.x₂). Then, by exploiting r^{ij} and r^{hk}, the interpolation is performed along the x₂ axis and the value p.v is finally determined.

Below follows the formal definition.

$${r}^{ij} \cdot v = \frac{{s}^{j} \cdot {x}_{1} - {r}^{ij} \cdot {x}_{1}}{{s}^{j} \cdot {x}_{1} - {s}^{i} \cdot {x}_{1}} {s}^{i} \cdot v + \frac{{r}^{ij} \cdot {x}_{1} - {s}^{i} \cdot {x}_{1}}{{s}^{j} \cdot {x}_{1} - {s}^{i} \cdot {x}_{1}} {s}^{j} \cdot v$$

$${r}^{hk} \cdot v = \frac{{s}^{k} \cdot {x}_{1} - {r}^{hk} \cdot {x}_{1}}{{s}^{k} \cdot {x}_{1} - {s}^{h} \cdot {x}_{1}} {s}^{h} \cdot v + \frac{{r}^{hk} \cdot {x}_{1} - {s}^{h} \cdot {x}_{1}}{{s}^{k} \cdot {x}_{1} - {s}^{h} \cdot {x}_{1}} {s}^{k} \cdot v$$

$$p \cdot v = \frac{{r}^{ij} \cdot {x}_{2} - p \cdot {x}_{2}}{{r}^{ij} \cdot {x}_{2} - {r}^{hk} \cdot {x}_{2}} {r}^{hk} \cdot v + \frac{p \cdot {x}_{2} - {r}^{hk} \cdot {x}_{2}}{{r}^{ij} \cdot {x}_{2} - {r}^{hk} \cdot {x}_{2}} {r}^{ij} \cdot v$$

The Tri-linear procedure is analogous: it just has three steps instead of two.

An SFA interpolating function captures non-linear moisture behaviors exploiting the knowledge about the hydrological fluxes. To this end, we rely on an Artificial Neural Network (ANN) that learns the soil behavior from a model-based soil simulator. ANN has chosen based on the following properties that meet the overall system goals:
Low resource consumer: once trained off line, the ANNs are fast and require limited computational resources.
Powerful & Flexible: can provide accurate profiles (i.e., they properly capture non-linearities) even the grid layout includes a limited number of sensors.
Robust: ANNs are well-known to work well even in presence of dirty or missing data. This may happen when a sensor occasionally fails to provide the correct data.

More in details, we adopted a feed-forward ANN with one, fully connected, hidden layer. The input layer has one neuron for each sensor, the output layer has as many neurons as the number of points in P (See FIG. 7). The ANN profiling function, given the sensor measurements, returns the appropriate values for each profile point. The ANN learns the soil behavior from simulated data in an off-line phase which is carried out only once at the time of installation of the system. The learning process is sketched in FIG. 7. (1) The simulation model is calibrated using the soil texture of the field we are interested to monitor. Such data are assumed to be provided as input by the farmer. (2) Training data are generated running the simulator according to real weather conditions and simulated watering sessions. The simulator returns the moisture profile, and the sensor measurements taken from the simulator output according to the real sensor layout that must be provided by the farmer too. A 4-month period of hourly profiles turned out to be sufficient to train the system. (3) Once training data are available, the machine learning algorithm trains the ANN.

FIGS. 8a, 8b clarify the differences between the SFA and SFU approaches. The Figures show two profiles obtained from a grid of four sensors positioned on the corners. The values for the sensors are the same for the two profiles (top-left=−10 cbar, top-right=−300 cbar, bottom-left=−200 cbar, bottom-right=−300 cbar).

The profile of FIG. 8a is obtained applying the SFU bi-linear profile function, while the profile of FIG. 8b through the SFA ANN. The apparent difference in the upper right part results from the composition of two factors: (i) proximity to the surface (ii) in a non-irrigated area. This non-linear effect is only captured by the ANN. We emphasize that this is an extreme case: as already mentioned, the higher the number of sensors, the lower the errors due to non-linear behaviors that are local to the intra-sensor regions.

In one embodiment, a visualization module allows an easy exploitation of the (augmented) moisture profile, enabling effective soil moisture monitoring. Visualization is made up of two components selected according to the data types and the visualization goals:
Historical moisture trend: shows the variation of moisture along time. Independently from the profile dimensionality, a 2D stacked area chart is adopted.
Spatial moisture: shows the spatial distribution of moisture at a specific time. A 2D heat map or a 3D scatter plot is adopted depending on the profile dimensionality.

The two components are linked through interactive zooming when the user selects a specific time on the historical moisture trend, the corresponding spatial moisture at the time is shown.

Since the goal is to check that an ideal moisture profile is maintained, we defined five farming-specific moisture ranges expressed in cbar, each associated with a different color. For example, Dark blue [0, −30) and light blue [−30, −100) show heavily/slightly over-watered soil, respectively. The green interval [−100, −300) represents the ideal case. Finally, light red [−300, −1500) and dark red [−1500, −inf] are the two most dry intervals, and indicate a portion of under-watered soil.

FIG. 9 shows an example of a 2D profile. The historical moisture chart shows how the soil gradually dries out during the dry season. In May the soil is mostly wet while in June the first irrigations are necessary. It also should be noted how the single-wing dripper fails to completely eliminate the red area due to the presence of an unwatered region in the soil volume. For the chosen zoom date, the spatial moisture chart shows the moisture profile in detail. Each cell corresponds to a profile element. The following areas can be highlighted: (i) the region under the dripper where the effects of irrigation are apparent; (ii) the superficial portion away from the dripper which is very dry as it is not irrigated; (iii) the deep region which is less affected by atmospheric agents and in which constant moisture remains regardless of the effects of watering. The profile also highlights how the moisture spreads laterally thanks to the combined effect of the roots and the permeability characteristics of the soil.

FIGS. 10a, 10b shows an example of a 3D spatial moisture chart. Each sphere corresponds to a profile element. The semantic of colors is the same as in the 2D visualization. Spaces between spheres allow the inner side of the 3D profile to be explored, furthermore, to facilitate the analysis, the chart can be rotated along the three axes. The moisture gradient along the third dimension (along the orchard rows) is better highlighted by the third orientation of the chart and justifies the adoption of a 3D profile.

Starting from the spatial profile, many meaningful visualizations may be derived. FIG. 11a shows the profile “Variance” chart, wherein the moisture variance along a given period is reported. The lighter areas in the chart are those where moisture varies the most. Similarly, in a profile “Mean” chart (FIG. 11b) the average moisture along a given period is reported. The charts can help to answer many questions formulated by the domain experts involved in the project:
Identify the irrigated volume: this region of the profile is characterized by a high mean and variance of moisture.
Identify the volume where the roots are: in this region typically moisture drops faster due to the absorption action of the roots.
Analyze the water dynamics in the soil: if by increasing the quantity of water supplied to the soil in a time interval, the irrigated volume does not increase significantly, it means that the soil, due to its characteristics, disperses water in depth.

### Experimental Results

The system has been tested on the installations of the Agro.Big.Data.Science project (ABDS, http://agrobigdatascience.it/). Data have been collected during the irrigation season 2020-2021.

(May to September). Installations are taken from two orchards: one in the plains and one in the hills around Faenza, in the province of Ravenna (Italy).

The orchards were planted in 2010 as a self-rooting Hayward variety (A. chinensis var deliciosa), grafted in 2012 with Gold 3 (A. chinensis var chinensis).

Kiwifruit vines were spaced 2 m along the row and 4.5 m between rows. Different irrigation systems and sensor grid arrangements have been considered as shown in FIG. 12. Sensor data are collected once per hour.

It should be noted that the number of sensors that have been used is much higher than necessary, as we will show below. A grid with 12-15 sensors was needed to build the ground truth in order to carry out the tests. Profile accuracy is calculated as the Root Means Square Error (RMSE) between the profile values and the sensor values in the corresponding positions, for those sensors that are not used as profile input. In other words, we verify how well the profile approximates the moisture in positions where moisture is known to the tester but hidden from the system.

FIG. 13 shows the system performance varying the number of input sensors.

To better understand the advantage provided by the method according to the invention, we also highlighted the performance of traditional approaches based on a single sensor (i.e., the de facto standard) or on a column of 3 sensors at different depths. In order to extend the values of the single sensor to the entire soil volume, we must assume that the moisture is constant. Similarly, when a column of sensors is available, we assume that moisture is constant at the same depth in the soil.

Obviously, the accuracy varies based on where the sensor is placed. To make a fair comparison we put ourselves in the best solution, that is we chose the single sensor or the sensor line that minimized the RMSE. The same methodology was adopted to dispose of the sensors used to calculate the profile.

It is apparent that single sensor estimates are by far less accurate than the ones obtained by the profiling functions. Three sensors are sufficient to halve the RMSE. The 1-dimensional approach achieves slightly better results because it captures the different moisture at different soil depths but still fails in capturing spatial variations. The extent of these errors is not negligible since the optimal range for soil moisture for kiwi cultivation is [−100; −300] cbar.

RMSE for SFA and SFU gradually decreases as the number of sensors increases.

It should be noted that the bilinear profiling function can be computed for some sensor arrangements only due to the intrinsic geometrical constraints (the profile region must be partitioned in bounding rectangle/cube). The ANN profiling function always outperforms the bilinear one due to its capability to model non-linear behaviors. This supremacy is more evident when the number of sensors is limited and the intra-sensor distances are larger (see FIGS. 8a, 8b). It should be noted that, even with a single sensor, ANN-based profile overcomes the standard single-sensor baseline due to its capability to model soil behavior.

FIG. 14 shows the comparison results regarding the 2D case.

The abscissa indicates the number of the utilized sensors, the columns represent the related approach performance. It should be noted that SFU does not have the same number of columns as SFA. Indeed, because of its nature, some arrangements are not allowed (each sensor has to be part of a bounding rectangle/cube).

Moreover, the approaches Single sensor and Sensors column are represented with horizontal lines because they are not dependent on the number of utilized sensors (they can work only in their ad hoc configurations).

In both 2D and 3D cases the same considerations hold.

As expected, the worst approach is Single sensor. In fact, drip irrigation causes the moisture to vary a lot in just few centimeters of soil, and it is too greedy to believe that the profile assumes the same value in its entirety. Follows the Sensors column approach: all the others perform always better, except for SFA with one sensor. This is because SFA finds it difficult to generate a reasonable profile with just one sensor in input, and it propagates such a sensor value, with some little changes. Yet, as the number of utilized sensors increases, the SFA performance visibly improves, and there is no approach able to overcome it. As already explained above, the learning phase makes the SFA able to capture non-linear soil behaviors, building a more precise moisture profile. The only case in which SFA and SFU achieved the same performance is with nine sensors. That is because, although SFU performs a linear interpolation, the composition of several linear strokes approximates a nonlinear behavior. Thus, the more sensors we use, the more the non-linear soil behaviors are captured (increasing the performance).

SFA has been proven to achieve the best performance. Moreover, it is the only approach that can work with the preferred number of sensors, and arrangements.

For these reasons, SFA was used to conduct a sensors disposal analysis.

We recall that, given a precise number of utilized sensors, we evaluated the performance of all the possible sensors arrangements. In the results shown before (FIG. 13), fixed the number of sensors, we selected the best arrangement (the one that achieved the best performance). Instead, to conduct this analysis, we filtered the best five. Then, we counted how many times each sensor is present in such best arrangements. In regard to the 2D case, FIG. 14 illustrates the frequency with which each sensor is chosen among such best arrangements. The sensor right under the dripper (at 20 cm of depth) appears in all the configurations.

As a matter of fact, that specific location is where the soil moisture varies the most. The irrigation has a lot of impact, and the plant sinks likewise.

It highlights that this is the most important spot to take into account. Follows the sensor in the same horizon at 90 cm from the dripper, where the effect of the irrigation is void and the evaporation is high. With 80% of frequency, this suggests that effective moisture profiles should have information also about the counterpart. Finally, the most frequent sensors are present at 40 cm of depth.

FIG. 15 shows an example of system architecture. In one embodiment, the system may run in two distinct phases: online and offline.

Sensors S provide a discretized representation of soil behaviors. However, such representations are coarse due to the reduced number of sensors installed in real-world applications. To prevent heavy investment in sensor equipment, our goal is to produce a fine-grained representation of soil moisture starting from a small number of sensors. To do so, we leverage a profiling function that, given real-time sensor data, produces a soil profile.

As explained above, the profiling function can be optionally learned offline. Given a soil texture St, we leverage a soil simulator Ss (M. Bittelli, A. Pistocchi, F. Tomei, P. Roggero, R. Orsini, M. Toderi, G. Antolini, M. Flury, “CRITERIA-3D: A mechanistic model for surface and subsurface hydrology for small catchments”, 2011) to produce many soil moisture scenarios with respect to different watering and weather conditions. Finally, such scenarios are given as input to a machine learning algorithm ML that learns how the soil behaves. Simulation and learning are heavy computations that are run (offline) in big data and cloud environments.

FIG. 16 shows an embodiment variant of the system architecture of FIG. 15.

Sensors S provide a discretized representation of soil behaviors. Given a Soil texture St, a Soil simulator Ss simulates the behavior of the soil taking into account atmospheric agents and various irrigation patterns. A dataset Ds of simulated data is extracted from the Soil simulator Ss. This dataset Ds is then used to train the interpolation via a neural network (SFA approach).

In one embodiment, the data collected by the sensors may also used to adapt the neural network to the behavior of the soil under examination through advanced machine learning techniques (continuous learning). In some cases, in fact, the Soil simulator Ss is not able to reproduce all the specificities of the soil (e.g., cracking and bedding).

When the neural network reaches a desired level of accuracy, it can be used to replace the linear interpolation.

It should be noted that, in one embodiment of the proposed method, the SFU interpolation (which does not need training) allows to extract the quantity profile immediately after having installed the sensors. The SFA interpolation, in fact, may run while SFU interpolation is in operation, as shown in the diagrams of FIGS. 15 and 16.

Therefore, according to one embodiment of the method, there is no need to provide for a data collection period before starting the monitoring system, that would cause a delay in the quantity monitoring.

It is remarked that (i) the offline phase is optional since statistical functions can be adopted for profiling, and (ii) once a profiling function is learned for a given soil texture, the same function can be adopted in similar soil textures without further training. The difference in accuracy between profiling functions has been shown above.

As mentioned above, in one embodiment, gypsum block sensors are used to measure soil moisture. Gypsum block sensors use two electrodes placed into a small block of gypsum to measure soil moisture in terms of soil water tension.

It is remarked that the proposed method and system may be based on the use of only one type of sensors, without the need for physical and manual soil sampling. The number of sensors is small (for example, 12 sensors).

The proposed method and system allow to monitor a small region of the soil and therefore to know the exact behavior of a plant, rather that of soil in the order of hectares of land.

The combination of the two aspects above allows to obtain a more precise profile compared to the known methods. For example, FERSCH B ET AL: “Synergies for Soil Moisture Retrieval Across Scales From Airborne Polarimetric SAR, Cosmic Ray Neutron Roving, and an In Situ Sensor Network”, WATER RESOURCES RESEARCH, vol. 54, no. 11, 23 Nov. 2018, discloses a sensor grid being deployed in a 70 m×70 m grid to monitor soil water and applying a profiling function to a finer grid, interpolated to a 5 m×5 m grid. However, in the 5 m×5 m grid, the sensors are 1 m mutually distanced, so too far for allowing a precision monitoring of the soil moisture in the soil volume occupied by the plant root apparatus.

In addition, it should be noted the proposed monitoring method considers the position of all the sensors of the grid, and this allows modeling how measurements of adjacent sensors influence each other, producing a more accurate model.

Depending on the offline/online phases, the software architecture may be composed as follows. The online phase runs in-situ (on edge) and is based, for example, on a Python implementation. Collected sensor data are both processed in real-time and forwarded to the remote environment. The offline phase runs on remote cloud-based open-source frameworks supporting storage, simulation, and machine learning. The cloud environment provides extensive resources necessary for both simulation and machine learning.

In summary, the methods and systems disclosed above may advantageously be employed:
(a) in agriculture, to create a moisture profile that is representative of the whole soil volume;
(b) in geology, to monitor the quantity of water in the ground for the prediction of landslides and more generally of hydro-geological risk;
(c) in construction, to monitor the quantity of water in a material to study its behavior and dynamics. For example, in order to predict the drying times of the concrete during the curing phase.

A person skilled in the art may make changes and adaptations to the embodiments of the method and systems according to the invention and replace elements with others, which are functionally equivalent, in order to satisfy contingent needs, without departing from the scope of the following claims. All of the features described as belonging to a possible embodiment can be achieved irrespectively of the other described embodiments.

What is claimed is:

1. A method for estimating a quantity profile of a volume of soil, comprising:
placing in the soil volume a bi-dimensional or three-dimensional grid of quantity sensors, wherein each quantity sensor of the grid is identified by a position represented by three-dimensional coordinates with respect to a center of the soil volume;
acquiring, in a same instant of time, a quantity value from each quantity sensor of the grid, so as to obtain a bi-dimensional or three-dimensional grid of real quantity values; and
applying a profiling function to the quantity value grid to obtain a fine grained quantity profile defined by a bi-dimensional or three dimensional extended grid of quantity values, each value of the extended grid being associated with a position represented by three-dimensional coordinates with respect to the center of the soil volume, wherein the number of elements of the extended grid of quantity values is much greater than the number of quantity sensors.

2. The method of claim 1, wherein the profiling function is a linear interpolation function between pairs of real quantity values.

3. The method of claim 2, wherein the linear interpolation function is applied between the pairs of real quantity values along each axis of the quantity value grid, independently of other axes.

4. The method of claim 2, wherein the linear interpolation is a bi-linear or a three-linear interpolation.

5. The method of claim 4, wherein each interpolated value of the extended grid is calculated considering a rectangle or parallelepiped whose vertices are the real quantity values closest to the point of the interpolated value and calculating a first linear interpolation between the pairs of real quantity values along a first dimension of the rectangle or parallelepiped, in order to obtain a pair of interpolated quantity values, a second linear interpolation along a second dimension of the rectangle or parallelepiped between the pairs of interpolated quantity values, and a possible third linear interpolation along a third dimension of the parallelepiped.

6. The method of claim 1, wherein the profiling function is a non-linear function that takes into account characteristics of the soil.

7. The method of claim 6, wherein the profiling function is implemented by a machine learning algorithm.

8. The method of claim 7, wherein the machine learning algorithm includes:
calibration of a numerical model that simulates hydrogeological flows of the soil;
generation of a training data set with the same grain of the profile to be approximated, the training data set being based on many different weather and irrigation conditions to fully capture soil dynamics; and
learning of a profiling function that maps real moisture values obtained from sensors into a fine grid moisture profile.

9. The method of claim 1, wherein soil quantity is one of: moisture, temperature, electrical conductivity, Nitrate concentrations.

10. A system for estimating a quantity profile of a volume of soil, comprising:
a bi-dimensional or three-dimensional grid of quantity sensors positionable in the soil volume to detect the value of the quantity, wherein each quantity sensor of the grid is identified by a position represented by three-dimensional coordinates with respect to a center of the soil volume; and
a processing unit operatively connected to the grid of quantity sensors, the processing unit being programmed to acquire, in a same instant of time, a quantity value from each quantity sensor of the grid, so as to obtain a bi-dimensional or three-dimensional grid of real values of the quantity, and to apply a profiling function to the quantity value grid to obtain a fine grained quantity profile defined by a bi-dimensional or three dimensional extended grid of quantity values, each value of the extended grid being associated with a position represented by three-dimensional coordinates with respect to the center of the soil volume, wherein the number of elements of the extended grid of quantity values is much greater than the number of quantity sensors.

11. The system of claim 10, wherein the quantity is soil moisture, and wherein the sensors are Gypsum blocks or Watermarks sensors.
